(19) United States
(12) Patent Application Publication
(10) Pub. No.: US 2006/0034884 A1
(43) Pub. Date: Feb. 16, 2006

(54) Coated medical device having an increased coating surface area

(76) Inventor: Eric B. Stenzel, Tuam (IE)

Correspondence Address: JONES DAY, 222 EAST 41ST ST, NEW YORK, NY, 10017, US

(21) Appl. No.: 10/916,109

(22) Filed: Aug. 10, 2004

(51) Int. Cl. A61F 13/00

(52) U.S. Cl. 424/422

(57) ABSTRACT

The present invention is directed to a coated medical device for delivering a biologically active agent to a body tissue such as a body lumen, said coated medical device having an increased coating surface area for adjusting the release rate of a biologically active agent, such as a drug, from the coating. The medical device has a coating comprising an outer surface having a surface area and capable of being in direct contact with the body tissue, and a plurality of indentations in the outer surface of the coating. The surface area of the coating outer surface is therefore greater than the surface area of the coating outer surface absent the indentations. The present invention is also directed to a method for making a medical device comprising forming a coating comprising a polymer and a biologically active agent on a surface of a medical device, wherein the coating comprises an outer surface capable of being in direct contact with body tissue, and increasing the surface area of the outer surface by forming indentations on the outer surface of the coating.

### FIELD OF THE INVENTION

The present invention relates generally to implantable medical devices. More specifically the present invention relates to a coated medical device having an increased coating surface area for adjusting the release rate of a biologically active agent, such as a drug, from the coating. The surface area of the coating's outer surface, or surface that is capable of directly contacting body tissue, is increased by forming indentations in the outer surface of the coating. The increased surface area provides more surface area through which the biologically active agent in the coating can be delivered to body tissue such as a body lumen. The invention is also directed to a method for manufacturing such a coated medical device.

### BACKGROUND OF THE INVENTION

A variety of medical conditions have been treated by introducing an insertable or implantable medical device such as a stent, catheter or vena cava filter having a coating for release of a biologically active agentl into body tissue, such as a body lumen of a patient. For example, various types of drug-coated stents have been used for localized delivery of drugs to a body lumen. See, e.g., U.S. Pat. No. 6,099,562 to Ding et al. These coatings provide the medical devices with certain advantages. Coatings containing antimicrobial agents have been applied to medical device surfaces to prevent infection. For example, U.S. Pat. No. 6,468,649 to Zhong et al. teaches an implantable medical device having a substrate with a hydrophilic coating composition to limit in vivo colonization of bacteria and fungi. Also, coatings containing therapeutic agents have been applied to stent surfaces because it is believed that such coatings help treat or prevent restenosis. For example, U.S. Pat. No. 6,258,121 to Yang et al. discloses a stent having a polymeric coating for controllably releasing an included active agent such as taxol, to inhibit restenosis following angioplasty.

Various methods are known in the art for coating medical devices. These include spray coating a composition of a biologically active agent and one or more polymers and solvents onto the surface of the medical device or dipping the medical device into the coating composition.

Once the medical device has been coated, it is often desirable to control the release rate of the biologically active agent from the coating into the body tissue. If the biologically active agent is released or delivered into the body tissue too quickly, the effect on the patient may be greater or more sudden than desired. Conversely, if the rate of release of the biologically active agent is too slow, the agent may not have the desired effect on the patient, and the efficacy of the agent will be lost or diminished.

Therefore, when a biologically active agent whose dosage or release rate must be controlled is contained in the coating of a medical device, it is important that the amount of the agent released over time be accurately predicted and controlled. The issue of effectively controlling the rate of release of the agent from the coating into body tissue, such as a body lumen has been addressed in the art. For example, U.S. Pat. No. 6,562,065 B1 to Shanley discloses an “expanding cage” stent design, comprising a stent structure that can be expanded using axial slots and ductile hinges. However, such complex configurations are often costly and difficult to manufacture. Furthermore, Shanley doesn't actually address the issue of drug release rates or discuss how a drug's release rate can be affected by expanding the stent structure and the surface area of the stent available for exposure to the body lumen.

Thus, it is desirable to have efficient and cost-effective methods of adjusting or controlling the rate of release of a biologically active agent from a coating disposed on a medical device, i.e. providing the coating with a desired release profile.

### SUMMARY OF THE INVENTION

The present invention is directed to a medical device having a surface upon which a coating is disposed. The coating, which has an outer surface having a surface area, comprises a biologically active agent and a polymer. Also, the outer surface of the coating is capable of being in direct contact with body tissue. The release or delivery rate of the biologically active agent from the coating is controlled or adjusted by including a plurality of indentations in the coating outer surface. The inclusion of such indentations allows the surface area of the coating outer surface to be greater than the surface area of the coating outer surface absent the indentations. In addition to increasing the surface area of the coating, the indentation process also provides a means of accessing the drug that is held deeper in the polymer compound. The increased surface area of the coating outer surface allows a greater amount of the biologically active agent in the coating to be released from the coating over a given period of time. Also, by adjusting the size or number of the indentations, the release or delivery rate of the biologically active agent from the coating can be adjusted or controlled.

In one aspect, the present invention is directed to a medical device such as a stent, for delivering a biologically active agent to a body tissue, such as a body lumen, said device comprising a device surface and a coating disposed on at least a portion of said device surface; wherein said coating comprises the biologically active agent and a polymer; and wherein said coating comprises (a) an outer surface having a surface area and capable of being in direct contact with said body tissue; and (b) a plurality of indentations in said coating outer surface; and wherein the surface area of the coating outer surface is greater than the surface area of the coating outer surface absent the indentations. The surface area of the coating outer surface may allow a greater amount of the biologically active agent in the coating to be released from the coating over a given period of time.

In one embodiment of the present invention, the biologically active agent of the present invention may comprise paclitaxel, a derivative of paclitaxel or an analogue of paclitaxel. The polymer may comprise polystyrene. In other embodiments of the present invention, the indentations in the coating outer surface may be a cross-section of any shape, such as the shape of a triangle or a rectangle. The indentations may or may not extend through the entire thickness of the coating, and they may or may not be of uniform size or shape. In another embodiment of the present invention, the coating may comprise two or more layers, or two or more biologically active agents, wherein the two or more layers each comprise the biologically active agent. In yet another embodiment, each layer may comprise more than one biologically active agent.

The present invention is also directed to a method for making a medical device comprising: (a) forming a coating comprising a polymer and a biologically active agent on a surface of the medical device; wherein the coating comprises an outer surface capable of being in direct contact with body tissue; and (b)increasing the surface area of the outer surface by forming indentations in the outer surface of the coating. The indentations may be formed in several ways, including removing portions of the coating or pricking the coating. In one embodiment, pricking of the coating may be conducted by applying to the coating outer surface an apparatus comprising one or more sharp protrusions, such as a screw or knife or any other sharp object, or such as a rolling wheel having an outer surface, said outer surface having thereon a plurality of spikes.

In another embodiment, the present invention is directed to a stent comprising a surface for delivering a biologically active agent to a body tissue, and a coating disposed on at least a portion of said stent surface, wherein said coating comprises the biologically active agent and a polymeric material, wherein said coating comprises (a) an outer surface having a surface area and capable of being in direct contact with said body tissue; and (b) a plurality of indentations in said outer surface; wherein the surface area of the coating outer surface is greater than the surface area of the coating outer surface absent the indentations; and wherein the biologically active agent comprises paclitaxel, a derivative of paclitaxel or an analogue of paclitaxel.

In another embodiment, the surface area of the coating outer surface allows a greater amount of the biologically active agent in the coating to be released from the coating over a given period of time than the amount of biologically active agent that would be released from the coating absent the indentations. The polymeric material preferably comprises a polystyrene.

### BRIEF DESCRIPTION OF THE DRAWINGS

FIG. 1 shows a cross-sectional view of a coated medical device of the present invention in which the coating has been applied to the surface of the medical device but indentations have not been made in the outer surface of the coating.

FIG. 2 represents an embodiment of the present invention in which the coating is indented by using a rolling spiked wheel, which is applied uniformly to the surface of the coating on the medical device.

FIGS. 3(a)-(b) represent embodiments of the invention in which the indentations are formed by pressing a sharp object such as a screw into the surface of the coating.

FIGS. 4(a)-(c) represent embodiments of the present invention in which the indentations do not extend through the entire thickness of the coating.

FIG. 5 represents an embodiment of the present invention in which the indentations in the outer surface of the coating are not of uniform shape or size.

FIGS. 6(a)-(c) represent various embodiments of the present invention in which the indentations have various shapes.

FIGS. 7(a)-(b) represent embodiments of the present invention in which the indentations extend through the entire thickness of the coating.

FIGS. 8(a)-(b) and FIGS. 9(a)-(b) represent embodiments of the present invention with indentations of various shapes and sizes.

FIGS. 10(a)-(b) represent embodiments of the present invention in which the coating is comprised of more than one layer.

FIG. 11 is a scanning electron microscope image of a stent that has been prickled with one indentation in each of two struts.

FIG. 12 is a scanning electron microscope image of a stent that has been prickled with six indentations.

FIG. 13 is a scanning electron microscope image of a stent that has been prickled with five indentations.

FIG. 14 is a graph showing the effect of prickling on drug release.

FIG. 15 is scanning electron microscope image of three spring loaded probes placed side by side.

### DETAILED DESCRIPTION OF THE INVENTION

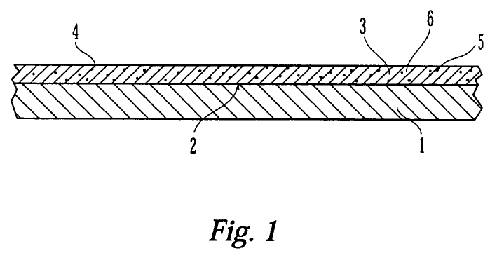

The medical devices of the present invention have a surface that is coated with a coating. FIG. 1 shows a cross-sectional view of a portion of a medical device 1 having a surface 2. Disposed on at least a portion of the medical device surface 2 is a coating 3 having an outer surface 4 that is capable of being in direct contact with body tissue, i.e. the outer surface of the coating refers to the surface of the coating that is capable of being directly exposed to body tissue. The coating 3 comprises a biologically active agent 5 and a polymer 6. In the embodiment shown in FIG. 1, indentations have not yet been made in the outer surface 4 of the coating 3. In order to achieve a coating having indentions in the outer surface of the coating, a coating composition comprising biologically active agent(s) and a polymer is obtained. This composition can include a solvent to dissolve or suspend the polymer and/or biologically active agent(s). The coating composition is applied to the surface 2 of the medical device 1. The coating composition can be applied to the surface 2 of the medical device 1 in a number of ways. Any known methods typically used in the art for coating medical devices can be used to apply the coating composition to the surface 2 of the medical device 1. One such preferable method is spray coating the coating composition onto the surface 2. Other preferred methods include dipping the medical device into the coating composition, application of the coating composition to the surface of the medical device by electrostatic means or air suspension, electrohydrodynamic coating, screen printing and condensation coating.

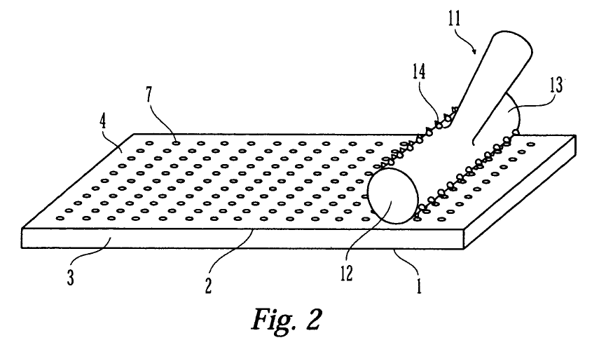

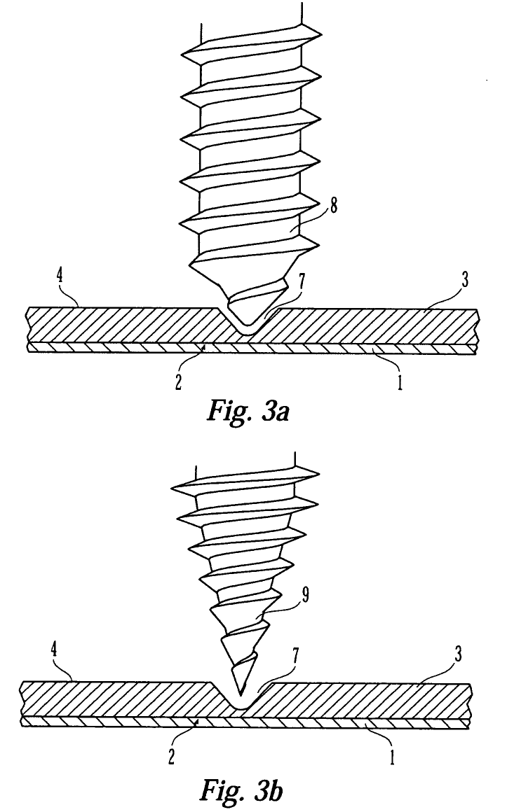

After the coating composition is applied to the medical device surface 2, a coating 3 is allowed to form. For example, the polymer in the coating may be allowed to cure to form the coating. Thereafter, indentations are made in the outer surface 4 of the coating. Such indentations can be formed in a number of ways. For example, the indentations may be formed by “pricking” the coating outer surface with an object or instrument that is capable of moving or pushing the coating material apart to form an indentation or a puncture in the coating. Instruments suitable for forming such indentations include, without limitation, instruments comprising wheels, as shown in FIG. 2. The instrument 11 has a wheel 12 having a surface 13 that is covered with a plurality of spikes 14 in which the wheel 12 can be rolled over the coating outer surface 4 to form the indentations 7 by forming punctures in the coating 3 and pushing apart the coating material. Another example of a suitable instrument is a mechanical drill for forming holes or indentations 7 into the outer surface 4 of the coating 3, as shown in FIG. 3(a). Other suitable methods include, but are not limited to, using a laser and/or any sharp point to form holes or indentations 7 in the outer surface of the coating, as shown in FIG. 3(b).

Forming the indentations by pricking the coating outer surface is particularly suitable where the coating is comprised of a stiffer or relatively less flexible polymer. Such polymers are less likely to experience recoil that can possibly allow the indentation to close upon itself. Examples of stiffer or relatively less flexible polymers are silicones or polymers with an increased percentage of polystyrene, which makes the coatings less tacky and more rigid.

In contrast, when the coating comprises a more spongy or springy polymer, it is preferable that the indentations are created by removing coating material from the coating outer surface. Removing the coating material in order to form the indentations avoids the possibility that the flexible polymer material used in the coating can “spring” back and cause the indentations to close upon themselves.

Portions of the coating can be removed from the outer surface of the coating by a number of ways. For instance, a laser can be used to remove the coating. Also, abrasive methods such as grinding or the use of a knife or scalpel to cut pieces of known dimension out of the coating can be used. In addition, the indentations can be formed by using a mechanical device such as a knife, scalpel, nail or other sharp point to physically cut out portions of the coating outer surface to provide the indentations. The device can physically scoop out portions of the coating outer surface, such as with a biopsy tool or a scalpel. The device can be one having both a vertical element and a circular element, such as a screw or a drill. Using screwdrivers, drills or other such instruments can provide a more accurate way of controlling the depth of penetration of the coating, and hence the desired surface area increase that can be achieved.

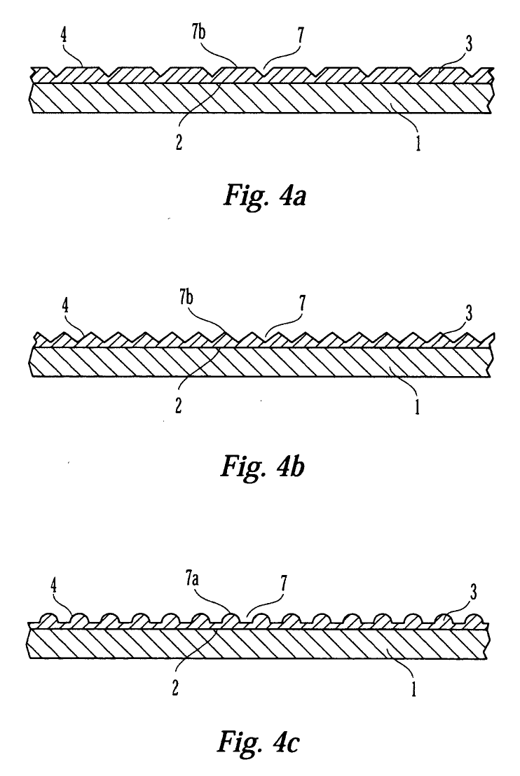

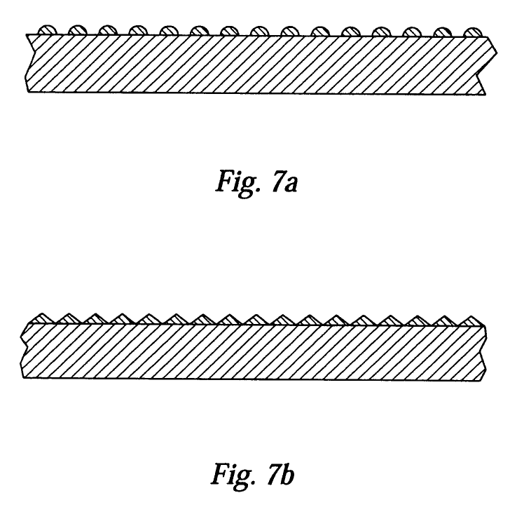

In another preferred embodiment of the present invention, the indentations are made in the coating with an instrument having a blunt tip, such that the end result is a coating outer surface 4 with protrustions, or bumps, 7a, as in FIG. 4(c). In two other preferred embodiments, illustrated in FIG. 4(a) and FIG. 7(b), instruments with sharper tips were used to achieve a more jagged surface with indentations 7 and ridges 7b protruding therefrom.

Furthermore, during the formation of the indentations in the outer surface of the coating, the medical device can be rotated or moved. This can speed up the formation of the indentations in the coating, and is advantageous when the indentations are applied using a rolling method. For example, if a drill or other mechanical device is used to push the coating inward or to remove a portion of the coating surface in order to form the indentations, such device can more effectively and efficiently form the indentations by the application of rotational force in addition to vertical linear force. Additionally, if the indentations are formed by applying a rolling wheel to the surface of the coating, as shown in FIG. 3, the act of rolling the wheel will naturally allow for a faster application of a greater number of indentations, in a more uniform and evenly-spaced manner, to the surface when the device is rotated. This leads to more efficient application of indentations, as well as more uniform indentation size and predictability of increase in surface area as a result of the indentations.

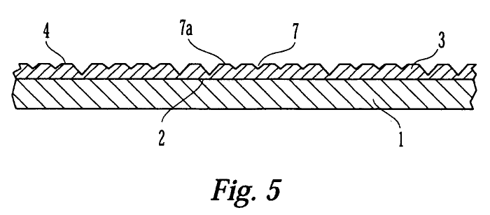

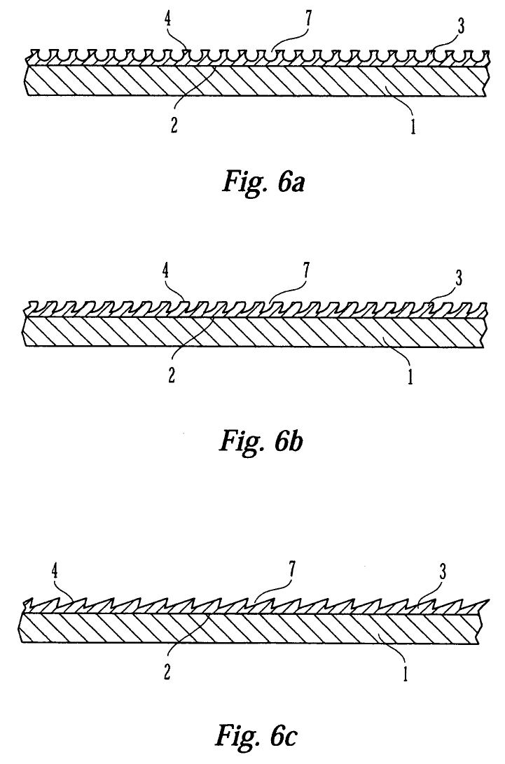

The indentations that are formed in the outer surface of the coating may have uniform dimensions or varying dimensions. FIGS. 4(a) through 4(c) illustrate embodiments in which the indentations 7 are of a uniform dimension. In contrast, FIG. 5 shows an embodiment in which the indentations 7 in the coating outer surface 4 are of varying sizes. Moreover, any number of indentations may be provided in the outer surface of the coating. Also, the indentations can be of any shape or orientation. Further, the indentations can have various cross-sectional shapes. For example, the indentations can have a cross-section in the shape of a triangle or rectangle. The indentations may penetrate directly downward into the coating, or they may be oblique or slanted. They may be pointed or conical in shape, spherical or blunt-tipped. FIGS. 6(a) through 6(c) show several preferred embodiments of the present invention in which the indentations are of varying shape and orientation.

More specifically, FIG. 6(a) shows an embodiment in which the indentations 7 are rounded. FIGS. 6(b) and 6(c) show embodiments wherein the indentations 7 are oblique. In addition, in certain embodiments such as those shown in FIGS. 4(a) through 4(c) and FIGS. 6(a) through 6(c), the indentations 7 do not extend through the entire thickness of the coating 3 to the surface of the medical device 2. In contrast, in other embodiments, such as those shown in FIGS. 7(a) and 7(b), the indentations 7 extend through the thickness of the coating 3.

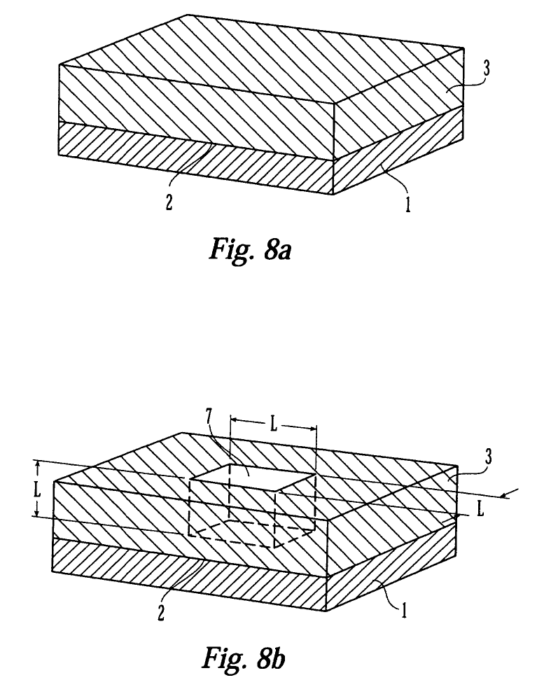

The inclusion of the indentations in the outer surface of the coating affects the release rate of the biologically active agent from the coating by increasing the amount of outer surface area of the coating that can be exposed to body tissue. More specifically, as shown in FIGS. 8(a) and 8(b), when removing portions of the coating from the surface from the coating on the medical device, the increase in surface area achieved by each indentation can be readily calculated.

For example, FIG. 8(a) is a view of an embodiment of the present invention, in which the coating 3 has been applied to the surface 2 of the medical device 1, but before the application of the indentations. The surface area of the coating has a value of X.

In FIG. 8(b), an indentation 7 with the dimensions of a cube has been cut out of the coating outer surface. The cube has a height, width and depth all of equal length L. Therefore, the surface area of the indented coating will now have a value that is equal to the surface area of the coating without indentation, i.e., X, plus the area of the sides of the cube; in other words, X+4(L²).

Similarly, in FIG. 9(a), where the indentation 7 is in the shape of a hemisphere having radius R, the surface area of the indented coating 3 will have a value that is equal to the surface area of the coating without indentation, i.e., X, plus the area of the curved part of the hemisphere, minus the area of the circle that lies in the plane of the surface prior to the formation of the indentation. The surface area of a sphere is 4ΠR², so the surface area of the indentation in such a case will be one-half of the surface area of a sphere, or 2ΠR². Therefore, the surface area of the indented coating will be X+2ΠR²−ΠR²=XΠR². Thus the surface area of the outer surface of the coating will increase by ΠR² due to the formation of the indentation.

In FIG. 9(b), where the indentation 7 is in the shape of a cylinder having radius R and height H, the surface area of the indented coating 3 will be the surface area of the circle that lies in the plane of the surface prior to the indentation, i.e., X or ΠR², plus the surface area of the wall of the cylinder. The surface area of the wall of the cylinder is 2ΠRH, so the surface area of the indented coating will be X+2ΠRH or ΠR²+2ΠRH. Thus the surface area of the outer surface of the coating will increase by 2ΠRH−ΠR².

Therefore, it is clear from the above that knowing the dimensions of the indentations can lead to more accurate prediction of the increase in surface area of the coating, and ultimately an increased accuracy in the predictability of release rates of the biologically active agent from the coating disposed on stents and other medical devices. An increase in surface area can also easily be calculated based on the known dimensions of the indentation instrument. For example, if the indentation instrument is a roller with spikes protruding therefrom, if the number and dimensions of each spike, and the surface area initially coated, are known, then the increase in surface area from indentation of the coated surface is easily calculated.

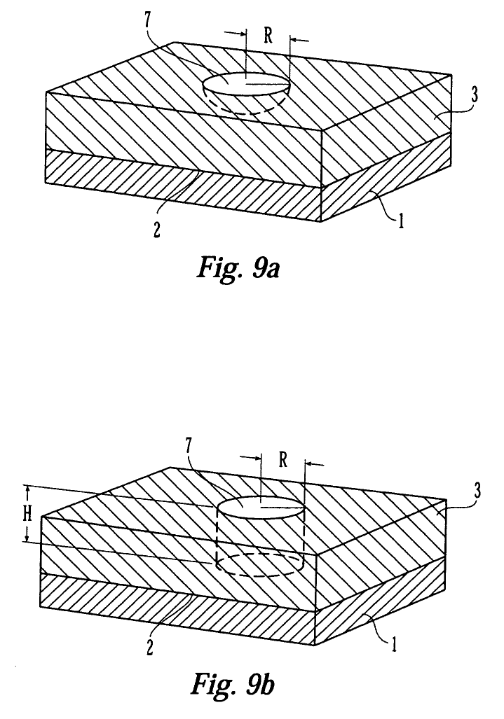

Moreover, the coatings of the present invention can comprise one or more layers, as shown in FIGS. 10(a) and 10(b). FIG. 10(a) depicts an embodiment in which the coating 3 comprises two layers, 3a and 3b. The indentations 7 in this case extend through both layers 3a and 3b. The coating layers may comprise different components, depending on the purpose of the coating composition and the desired composition of biologically active agents to be released into the body lumen, as well as the rate of release of each of the agents. FIG. 10(b) illustrates an embodiment of the present invention in which the coating surface comprises 2 layers, each of which comprises separate coating compositions. The indentations in this embodiment penetrate only one layer 3b. This leaves the lower layer 3a capable of being exposed to body tissue. Also, the indentations may go through one or more or all of the layers, either in part or in their entirety. Other embodiments involve variations in which a plurality of coatings are applied to the surface, and more than one coating is penetrated by indentation.

In the present invention, the term “medical device” can be used to refer to, without limitation, items such as catheters, stents, endotracheal tubes, hypotubes, filters such as those for embolic protection, surgical instruments and the like. Any device that is typically coated in the medical arts, and is capable of being inserted or implanted into the body of a patient, can be used in the present invention. The present invention is particularly useful in conjunction with local delivery of drugs or therapeutic substances on a stent within the vascular system. The invention may also be utilized in conjunction with drug delivery from balloon catheters or stents for use in other body lumens. The invention is particularly useful when utilizing a water soluble drug or therapeutic substance which tends to dissolve and migrate within a blood or other body fluid environment.

Examples of suitable medical devices for use with the present invention include stents, catheters, endotracheal tubes, hypotubes, filters such as those for embolic protection, surgical instruments and the like. Any device that is typically coated in the medical arts and is capable of being inserted or implanted into a body lumen for release of a biologically active material can be used in the present invention. The medical device preferably includes a body portion having an exterior surface defined thereon with the body portion being expandable from a first position, wherein the body portion is sized for insertion into the vessel lumen, to a second position, wherein at least a portion of the exterior surface of the medical device is in contact with the lumen wall. Most preferably, the medical device is a stent.

The term “coating composition” refers to any composition that is desired to be deposited upon the surface of a medical device, including those components that are to be later removed through methods such as evaporation. The components in the coating composition must be able to withstand temperature and pressure extremes associated with the methods used to apply them to the surface of the medical device and to withstand the pressure necessary to provide the indentations on the coating. Additionally, the components in the coating composition must be compatible with each other.

Preferably, the coating composition comprises a solvent, a polymeric material, and at least one biologically active agent. Upon evaporation of the solvent, a polymeric coating is formed. Preferred solvents include organic solvents such as toluene, tetrahydrofuran (THF), chloroform, toluene, acetone, isooctane, 1,1,1-trichloroethane, dichloromethane, dimethyl acetamide (DMAC), methyl ethyl ketone and mixtures thereof. Of these, toluene and THF are most preferred.

The term “therapeutic agent” as used in the present invention encompasses drugs, genetic materials, and biological materials and can be used interchangeably with “biologically active material”. Non-limiting examples of suitable therapeutic agent include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti-thrombin antibodies, anti-platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, 2′-O-ester with N-(dimethylaminoethyl)glutamide hydrochloride salt, nitroglycerin, nitrous oxides, nitric oxides, antibiotics, aspirins, digitalis, estrogen, estradiol and glycosides. In one embodiment, the therapeutic agent is a smooth muscle cell inhibitor or antibiotic. In a preferred embodiment, the therapeutic agent is taxol (e.g., Taxol®), or its analogs or derivatives. In another preferred embodiment, the therapeutic agent is paclitaxel, or its analogs or derivatives. In yet another preferred embodiment, the therapeutic agent is an antibiotic such as erythromycin, amphotericin, rapamycin, adriamycin, etc.

The term “genetic materials” means DNA or RNA, including, without limitation, of DNA/RNA encoding a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as anti-proliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents, vasodilating agents, and agents which interfere with endogenous vasoactive mechanisms;
anti-oxidants, such as probucol;
antibiotic agents, such as penicillin, cefoxitin, oxacillin, tobranycin, rapamycin (sirolimus);
angiogenic substances, such as acidic and basic fibroblast growth factors, estrogen including estradiol (E2), estriol (E3) and 17-beta estradiol;
drugs for heart failure, such as digoxin, beta-blockers, angiotensin-converting enzyme (ACE) inhibitors including captopril and enalopril, statins and related compounds; and
macrolides such as sirolimus or everolimus.

Preferred biological materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogs, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl)glutamine, and 2′-O-ester with N-(dimethylaminoethyl)glutamide hydrochloride salt.

Other suitable therapeutic agents include tacrolimus, halofuginone, inhibitors of HSP90 heat shock proteins such as geldanamycin, microtubule stabilizing agents such as epothilone D, phosphodiesterase inhibitors such as cliostazole.

Other preferred therapeutic agents include nitroglycerin, nitrous oxides, nitric oxides, aspirins, digitalis, estrogen derivatives such as estradiol and glycosides.

In one embodiment, the therapeutic agent is capable of altering the cellular metabolism or inhibiting a cell activity, such as protein synthesis, DNA synthesis, spindle fiber formation, cellular proliferation, cell migration, microtubule formation, microfilament formation, extracellular matrix synthesis, extracellular matrix secretion, or increase in cell volume. In another embodiment, the therapeutic agent is capable of inhibiting cell proliferation and/or migration.

In certain embodiments, the therapeutic agents for use in the medical devices of the present invention can be synthesized by methods well known to one skilled in the art. Alternatively, the therapeutic agents can be purchased from chemical and pharmaceutical companies.

The polymeric material should be a material that is biocompatible and avoids irritation to body tissue. The polymeric materials that can be used in the coating composition of the present invention include: polyurethanes, silicones (e.g., polysiloxanes and substituted polysiloxanes), and polyesters. Also preferable as a polymeric material is styrene-isobutylene-styrene (SIBS). Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (etylene-propylene-diene) rubbers, fluorosilicones, polyethylene glycol, polysaccharides, phospholipids, and combinations of the foregoing.

### EXAMPLES

### Example 1

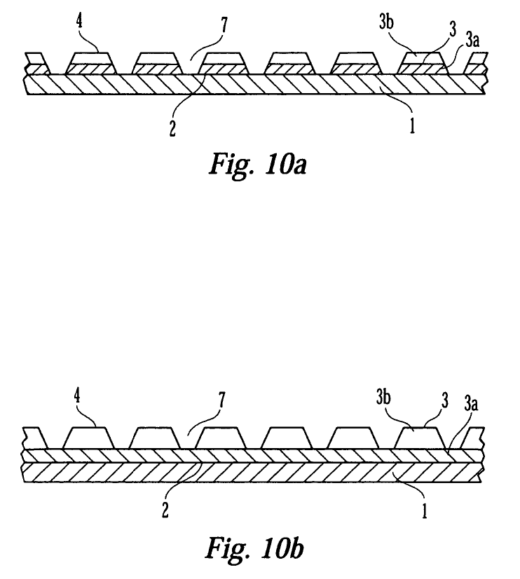

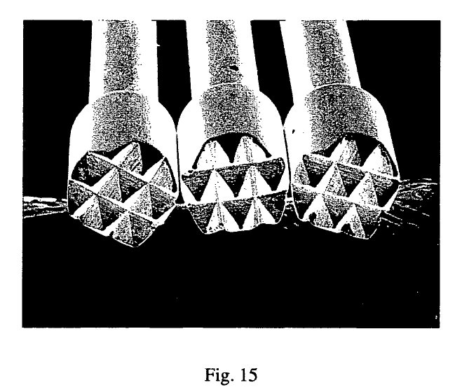

A stent coated with a paclitaxel and polymer formulation was first prepared using a standard coating process. The coated stent was then manually prickled using a needle tipped probe. The indentations were approximately the thickness of the coating layer and extended to the stent surface. To aid the manual prickling process, a fixture was manufactured where multiple spring loaded multi-point needle probes were aligned side by side. An example is shown in FIG. 15 that shows three spring loaded probes placed side by side. Each of the three probes in FIG. 15 has three rows of three needle tips each. FIG. 11 is an SEM image of the stent after prickling.

### Example 2

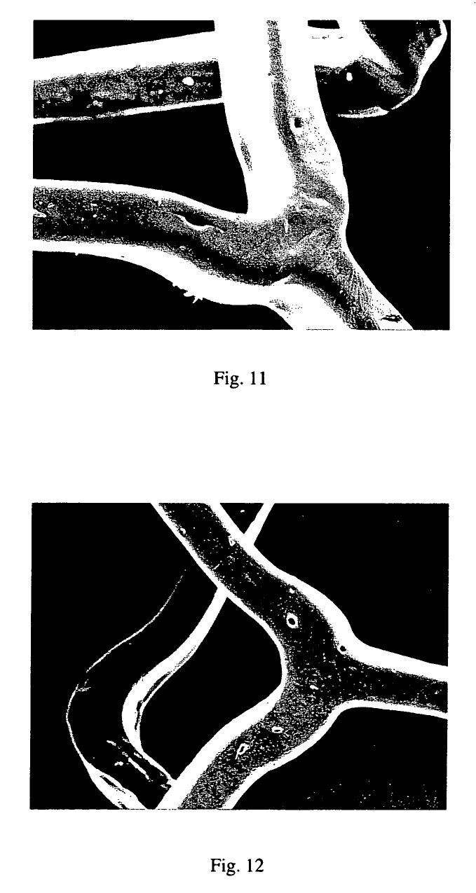

A stent coated with a paclitaxel and polymer formulation was first prepared using a standard coating process. The coated stent was then manually prickled using the needle tipped probe described in Example 1. The indentations were approximately the thickness of the coating layer and extended to the stent surface. FIG. 12 is an SEM image of the stent after prickling.

### Example 3

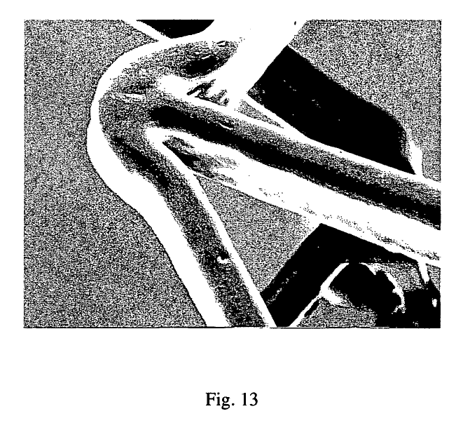

A stent coated with a paclitaxel and polymer formulation was first prepared using a standard coating process. The coated stent was then manually prickled using the needle tipped probe described in Example 1. The indentations were approximately the thickness of the coating layer and extended to the stent surface. FIG. 13 is an SEM image of the stent after prickling.

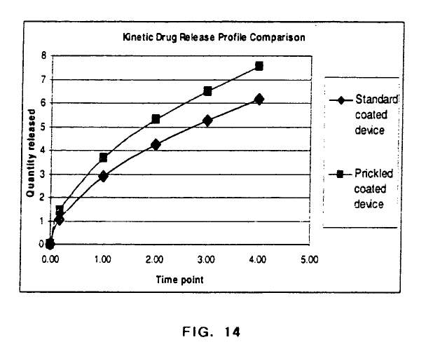

FIG. 14 is a normalized graph showing how prickling of the coating of the above three Examples affects the release profile of the drug, relative to coated stents whose coatings were not prickled. The coated stents whose coatings were prickled released more drug over a given time period.

It will be appreciated by those skilled in the art that while the invention has been described above in connection with particular embodiments, the invention is not necessarily so limited and that numerous other embodiments, examples, uses, modifications and departures from the embodiments described herein may be made without departing from the inventive concept. Also, the references mentioned herein are incorporated by reference in their entirety.

I claim:

1. A medical device for delivering a biologically active agent to a body tissue, said device comprising a device surface and a coating disposed on at least a portion of said device surface;
wherein said coating comprises the biologically active agent and a polymer; and
wherein said coating comprises (a) an outer surface having a surface area and capable of being in direct contact with said body tissue; and (b) a plurality of indentations in said coating outer surface; and
wherein the surface area of the coating outer surface is greater than the surface area of the coating outer surface absent the indentations.

2. The device of claim 1, wherein the surface area of the coating outer surface allows a greater amount of the biologically active agent in the coating to be released from the coating over a given period of time.

3. The device of claim 1, wherein the body tissue is a body lumen.

4. The device of claim 1, wherein said medical device is a stent.

5. The device of claim 1, wherein said biologically active agent comprises paclitaxel, a derivative of paclitaxel or an analogue of paclitaxel.

6. The device of claim 1, wherein said polymer comprises polystyrene.

7. The device of claim 1, wherein the indentations have a cross-section in the shape of a triangle or a rectangle.

8. The device of claim 1, wherein the indentations do not extend through the entire thickness of the coating.

9. The device of claim 1, wherein the indentations are of a uniform size and shape.

10. The device of claim 1, wherein the coating comprises more than one layer.

11. The device of claim 10, wherein the coating comprises two layers.

12. The device of claim 11, wherein the two layers each comprise the biologically active agent.

13. The device of claim 1, wherein the coating comprises at least one additional biologically active agent.

14. A method for making a medical device comprising:
(a) forming a coating comprising a polymer and a biologically active agent on a surface of the medical device; wherein the coating comprises an outer surface capable of being in direct contact with body tissue; and
(b) increasing the surface area of the outer surface of the coating by forming indentations in the outer surface of the coating.

15. The method of claim 14 wherein the indentations are formed by removing portions of the coating.

16. The method of claim 14, wherein the indentations are formed by pricking the coating.

17. The method of claim 14, wherein the pricking is conducted by applying to the coating outer surface an apparatus comprising one or more sharp protrusions.

18. The method of claim 17, wherein the apparatus comprises a rolling wheel having an outer surface, said outer surface having thereon a plurality of spikes

19. A stent comprising a surface for delivering a biologically active agent to a body tissue, and a coating disposed on at least a portion of said stent surface;
wherein said coating comprises the biologically active agent and a polymeric material;
wherein said coating comprises (a) an outer surface having a surface area and capable of being in direct contact with said body tissue; and (b) a plurality of indentations in said outer surface;
wherein the surface area of the coating outer surface is greater than the surface area of the coating outer surface absent the indentations; and
wherein the biologically active agent comprises paclitaxel, a derivative of paclitaxel or an analogue of paclitaxel.

20. The stent of claim 19, wherein the surface area of the coating outer surface allows a greater amount of the biologically active agent in the coating to be released from the coating over a given period of time than the amount of biologically active agent that would be released from the coating absent the indentations.

21. The Stent of claim 19, wherein the polymeric material comprises a polystyrene.
